(19) United States
(12) Patent Application Publication
(10) Pub. No.: US 2008/0312559 A1
(43) Pub. Date: Dec. 18, 2008

(54) Method and Apparatus for Performing Gastric Bypass Surgery

(76) Inventors: Albert N. Santilli, Pepper Pike, OH (US); Gerald Marsh, Cleveland Heights, OH (US)

Correspondence Address: WAYNE D PORTER, JR;LAW OFFICES OF WAYNE D. PORTER, JR., 1370 ONTARIO STREET, SUITE 600, CLEVELAND, OH, 44113, US

(21) Appl. No.: 12/137,366

(22) Filed: Jun. 11, 2008

Related U.S. Application Data
(60) Provisional application No. 60/943,502, filed on Jun. 12, 2007.

(51) Int. Cl. A61B 5/103; A61M 29/02

(52) U.S. Cl. 600/593; 606/192; 604/99.04

(57) ABSTRACT

A specially configured esophageal-gastric tube can be inserted into a patient's esophagus with the tip of the tube in the region of an end-to-end anaotomosis connection. The joined members are occluded upstream and downstream of the anastomosis connection. Fluid is pumped into the occluded section through the esophageal-gastric tube until a desired pressure level is attained. The anastomosis connection then is checked for leaks. The invention also includes a bougie having an end portion made of a material suitable for resisting electro-cauterization temperatures.

### REFERENCE TO PROVISIONAL APPLICATION

The present application claims priority to U.S. provisional application Ser. No. 60/943,501, entitled Method and Apparatus for Performing Gastric Bypass Surgery, filed Jun. 12, 2007 by Gerald Marsh and Albert N. Santilli.

### BACKGROUND OF THE INVENTION

1. Field of the Invention

The invention relates to surgical procedures and, more particularly, to a method and apparatus that can be used to perform gastric bypass surgery.

2. Description of the Prior Art

While the present invention has application in various types of surgical procedures, it will be described in the context of gastric bypass surgery as used for the treatment of obesity. The most common gastric bypass procedure performed today is known as the Roux-en-Y gastric bypass procedure (RYGB). In the RYGB procedure, a six-inch to eight-inch incision is made that extends from the end of the sternum to just above the navel. More recently, the RYGB has been performed laparoscopically in order to minimize trauma, healing time and risk of infection. The stomach is completely divided into two unequal portions—a small upper pouch and a large lower gastric pouch (excluded stomach). The upper pouch typically measures less than about one ounce, preferably about one-half ounce, or 15 cc, while the excluded stomach remains generally intact and continues to secrete stomach juices that flow through the intestinal tract.

The small intestine is severed at a location distal of the duodenum or proximal of the jejunum. The severed end of the small intestine then is brought from the lower abdomen, behind the colon and the bypassed stomach, and joined with the upper pouch to form an end-to-end anastomosis created through a half-inch opening, also called the stoma. This rerouted segment of the small intestine is called the “Roux loop” and carries food from the upper pouch to the remainder of the intestines where the food is digested. The severed end of the segment of the duodenum that is part of the excluded stomach is connected to the Roux loop by means of an anastomotic connection. The connection is located approximately 100 cm from the stoma, and typically is made by using a stapling instrument. Prior to completion of the surgical procedure, a gastropexy commonly is performed to attach the excluded stomach to the abdominal wall or to the diaphragm, primarily to prevent the excluded stomach from being displaced within the abdominal cavity.

The RYGB procedure described permits digestive juices from the bypassed stomach, pancreas and liver to join the food stream from the small upper pouch and Roux loop to begin digesting the food. The remainder of the intestinal tract is not disturbed. Due to the small size of the upper pouch, patients are forced to eat at a slower rate and are satiated much more quickly, thereby reducing their caloric intake. Moreover, because the food enters the intestines directly, certain undesirable foods such as sweets create unpleasant feelings of nausea, diarrhea, nervousness, and sweating, which in turn discourages patients from developing or maintaining unhealthy eating habits. The RYGB procedure typically demonstrates a loss of at least 50% of excess body weight; approximately 60% of the patients will be able to maintain this weight loss for at least five years.

During the course of performing the RYGB procedure, it is necessary at various times to evacuate, decompress, and lavage the stomach. An important part of the procedure is to form a properly sized and shaped stoma and to create an effective anastomosis connection. Typically this is done by introducing a specially formed bougie into the gastric area through the esophagus. It also is necessary to pressure test the completed stoma in order to make certain that no leaks are present.

A problem in performing the RYGB procedure is that various separate tubes and instruments must be used to accomplish the noted tasks, thereby making the procedure more complex, time-consuming, and expensive than desired. Another problem is that it can be difficult to position tubes and instruments at a proper location or position within the esophagus or gastric pouch. Yet an additional problem is that it has not been possible to adequately pressure test the completed stoma because only low pressure testing procedures have been available. Yet an additional problem is that bougies used to form the stoma and to perform the anastomosis are not very durable. As a consequence, the bougies usually are suitable for one-time use, which increases the overall cost of the procedure. Desirably, a method and apparatus would be available to conduct gastric bypass surgery that would overcome the foregoing problems.

### SUMMARY OF THE INVENTION

In response to the foregoing concerns, the present invention provides a new and improved method and apparatus especially adapted for performing gastric bypass surgery. Apparatus according to the invention includes an esophageal-gastric tube that has an elongate first tube made of a flexible material such as latex, vinyl, silicone, or similar material that defines a first, hollow passageway. The tube has a tip end, a central portion, and a tapered entrance/exit end. The tip end is provided with openings that establish fluid communication with the passageway. A first syringe can be attached to the tapered entrance/exit end.

A thin, expansible balloon is disposed relatively close to the openings on the side of the openings closest to the central portion. The balloon is in fluid communication with a second, hollow passageway. The second passageway either is included as part of the wall that defines the central portion or it is part of a separate tube that is disposed within the passageway. The second passageway terminates in a branch tube that projects from the side of the first tube adjacent the entrance/exit end. A shutoff valve is disposed in the branch tube near the exposed end thereof. A second syringe is adapted to be connected to the branch tube. If desired, a number of radiographic markers can be disposed within the first tube, usually within the central portion. The markers are evenly spaced at intervals along the length of the tube.

The apparatus includes a bougie that has an elongate body portion and a specially formed tip. The body portion and the tip are formed of a fairly rigid, flexible substance such as high-temperature silicone. The tip is coated with a substance such as TEFLON fluoropolymer that will resist the high electro-cauterization temperatures experienced during formation of the stoma. Apparatus according to the invention also includes a clamp for occluding a desired portion of the Roux loop.

The method according to the invention includes the steps of providing a tube and positioning the tube within the esophagus with the distal end of the tube downstream of the stoma. The method includes the step of occluding the Roux loop downstream of the newly formed stoma and the end of the tube. The method includes the further step of establishing an airtight seal in the esophagus and thereafter pumping air through the tube. The pressure is increased so that a good test of the integrity of the various sutures and staples is obtained.

The foregoing and other features and advantages of the invention will be apparent from a review of the following description of the invention, taken together with the attached drawings.

### BRIEF DESCRIPTION OF THE DRAWINGS

FIG. 1 is a schematic view of a patient's stomach and small intestine after undergoing the RYGB procedure;

FIG. 2 is a view of an esophageal-gastric tube according to the invention with two syringes and an adapter in place;

FIG. 3 is a view similar to FIG. 2 in which the syringes and the adapter have been disconnected and moved apart for purposes of clarity of illustration;

FIG. 4 is an enlarged view of a tip end of the esophageal-gastric tube of FIG. 2;

FIG. is a schematic view of a bougie according to the invention; and

FIG. 6 is a schematic view of the esophageal-gastric tube of FIG. 2 being used during the course of performing a RYGB procedure.

### DESCRIPTION OF THE PREFERRED EMBODIMENT

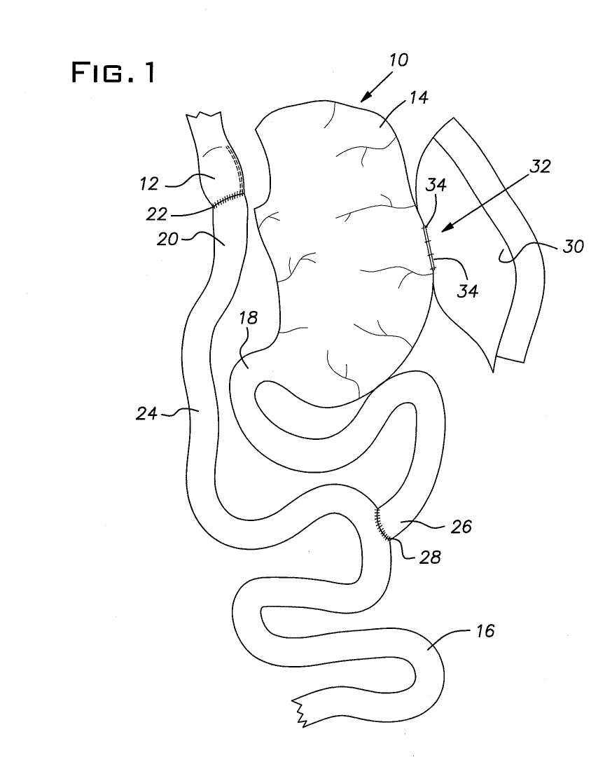

The RYGB procedure is illustrated in FIG. 1. The patient's stomach 10 is completely divided into two unequal portions—a small upper pouch 12 and a large lower gastric pouch 14 (or excluded stomach). The upper pouch 12 typically measures less than about one ounce, preferably about one-half ounce, or 15 cc, while the larger lower pouch 14 remains generally intact and continues to secrete stomach juices that flow through the intestinal tract.

The small intestine 16 is severed at a location distal of the duodenum 18 or proximal of the jejunum (not shown). A first severed end 20 of the small intestine then is brought from the lower abdomen, behind the colon and the bypassed stomach, and joined with the upper pouch 12 to form an end-to-end anastomosis 22 created through a half-inch opening, also called the stoma. This rerouted segment 24 of the small intestine is called the “Roux loop” and carries food from the upper pouch 12 to the remainder of the intestines where the food is digested. A second severed end 26 of the segment of the duodenum 18 that is attached to the lower pouch 14 of the stomach 10 is connected to the Roux loop 24 by means of an anastomotic connection indicated at 28. The connection 28 is located approximately 100 cm from the stoma 22, and typically is made by using a stapling instrument.

A gastropexy is performed, whereby the excluded stomach 14 is connected to the abdominal wall 30 by means of a marker/connector 32. Sutures 34 are passed through loops included as part of the marker/connector 30 and through the tissue of the adjacent pouch 14 and the abdominal wall 30. The marker/connector 32 thus serves as a fluoroscopic marker as well as a mechanical connector between the excluded stomach 14 and the abdominal wall 30. If it is necessary to insert an endoscope or a trochar into the excluded stomach 14, the endoscope or trochar can be inserted through the marker/connector 30 which will serve as a target as well as a means for holding the excluded stomach 14 in place as the endoscope or trochar is inserted therethrough.

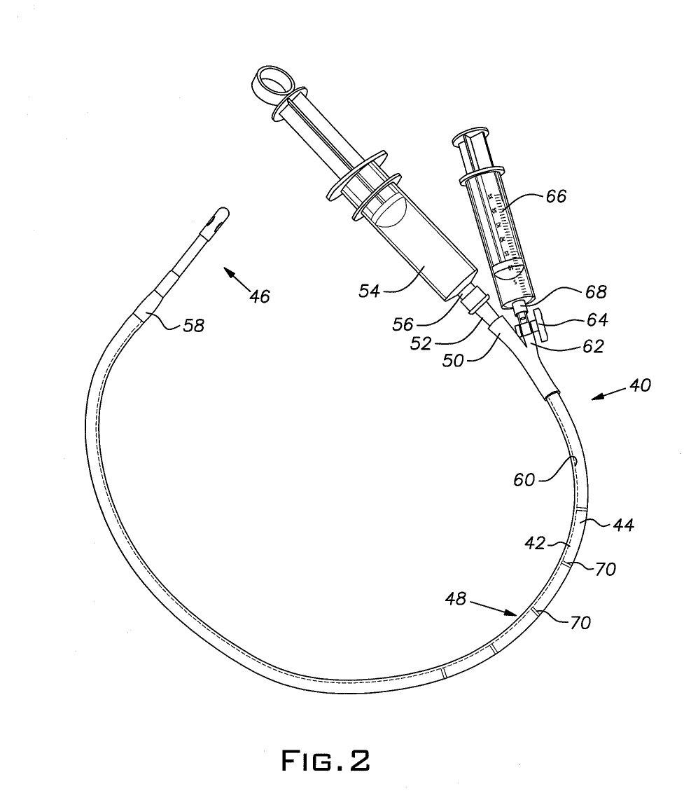

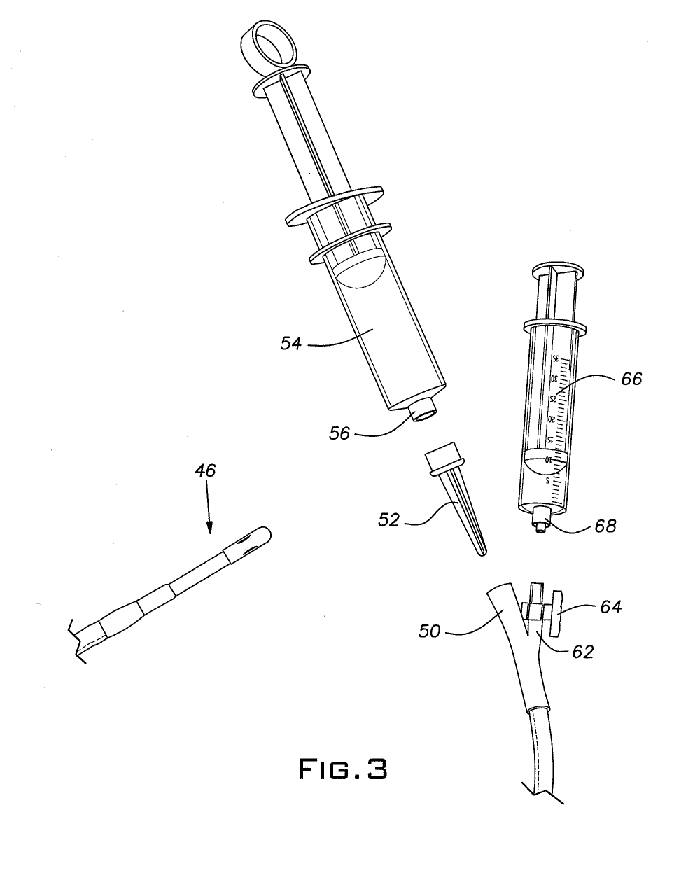

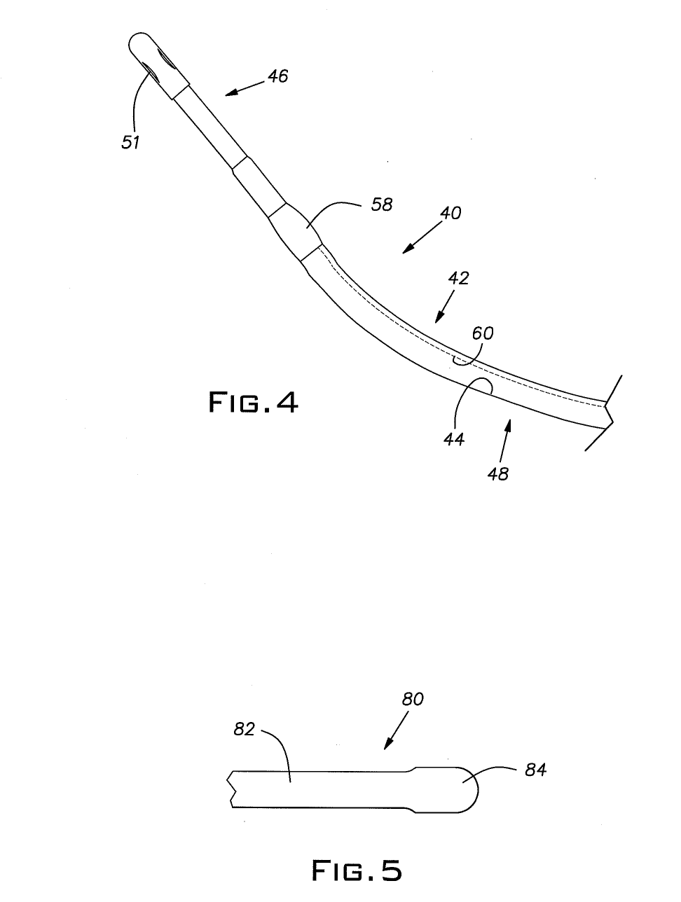

Referring now to FIGS. 2, 3, and 4, apparatus for use during the course of gastric surgery is indicated by the reference numeral 40. The apparatus 40 includes an esophageal-gastric tube that includes an elongate first tube 42 made of latex, vinyl, silicone, or similar material that defines a first, hollow passageway 44. The tube 42 has a tip end 46, a central portion 48, and a tapered entrance/exit end 50. The tip end 46 is provided with a pair of elongated openings 51 that are disposed on opposite sides of the end 46 and which establish fluid communication with the passageway 44. The tapered end 50 receives an adapter 52 to which a large syringe 54 (about 70 ml) can be attached by means of a luer slip connector 56. Typically, the first tube 42 will be provided in three sizes: 22 French (7.33 mm) for gastric bypass; 22 French and 24 French (7.33 mm and 6.0 mm, respectively) for pre-induction anesthesia; and 20 French and 22 French (6.67 mm and 7.33 mm, respectively) for oral and naso-gastric lavage and evacuation.

A thin, expansible balloon 58 is disposed relatively close to the openings 51 on the side of the openings 51 closest to the central portion 48. The balloon 58 is connected to the tube 42 by means of a second, hollow passageway 60. The second passageway 60 either is included as part of the wall that defines the central portion 48 or it is part of a separate tube that is disposed within the passageway 44. The passageway 60 terminates in a branch tube 62 that projects from the side of the first tube 42 adjacent the entrance/exit end 50. A shutoff valve 64 is disposed in the branch tube 62 near the exposed end thereof. A small syringe 66 (about 35 ml) is adapted to be connected to the branch tube 62 by means of a luer lock connector 68. If desired, a number of radiographic markers 70 can be disposed within the first tube 42, usually within the central portion 48. The markers 70 are evenly spaced at approximately 10 cm intervals along the length of the tube 42.

Referring now to FIG. 5, a bougie according to the invention is indicated by the reference numeral 80. The bougie 80 has an elongate body portion 82 and a specially formed tip 84. The body portion 82 and the tip 84 are formed of a substance such as high-temperature silicone. The tip 84 is coated with a substance such as TEFLON fluoropolymer that will resist the high electro-cauterization temperatures experienced during formation of the stoma 22.

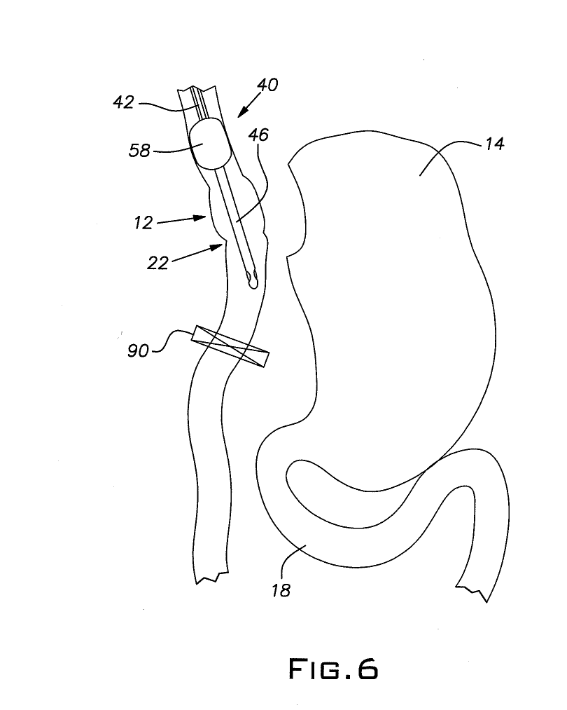

Referring to FIG. 6, the apparatus 40 according to the invention includes a clamp 90. The clamp 90 is indicated schematically, as it can be any type of commonly available clamp suitable for occluding conduits such as the small intestine. For example, an appropriately sized bulldog clamp or an occluding forceps would be usable provided it clamped the intestine such that fluid flow was prevented.

### Indications for Use and Features of the Apparatus 40

The apparatus 40 is used for positive air-leak detection during gastric bypass surgery, eliminating the need for added procedural time and expense. The apparatus 40 provides positive intra-operative air-leak detection for both open and laparoscopic gastric bypass procedures. It permits pre-operative gastric evacuation and pre-anesthesia induction esophageal occlusion. The apparatus 40 also permits intra-operative oro-gastric lavage and can be used for x-ray determination of lumen size and catheter position. The apparatus 40 also can be used as a stent during anastomosis.

The apparatus 40 features a lumen-occluding balloon 58 that is recessed to prevent back-flow during leak testing. The balloon 58 preferably is spaced about 9.0 cm behind the end of the tip 46. The tip 46 is soft in order to decrease trauma to mucosal tissue. The tip 46 is tapered for easier passage through the stoma anastomosis 22. The apparatus 40 preferably is made of hypoallergenic silicone construction to aid in manipulation and placement. The apparatus 40 reduces incidents of false channel and bowel perforation. The optional radiopaque markers 70, preferably at 10 cm intervals, provide x-ray verification. The apparatus 40 also stents the bowel lumen to aid in accuracy when sizing the bypassed pouch. Because the apparatus 40 performs multiple functions such as lavage and pressure testing, there no longer is a need to use separate tubes and instruments to accomplish the previously described tasks.

The bougie 80 is used for intra-operative gastric decompressions and as a stent during anastomosis. It permits intra-operative gastric lavage and may be used to determine the size of the anastomosis.

The bougie 80 has a specially designed tip 84 that resists the effects of electro-cauterization that typically occurs during the course of an anastomosis procedure. Preferably the tip 84 is coated with a heat-resistant coating such as TEFLON fluoropolymer or any other suitable temperature-resistant or temperature-dissipating material. The body portion 84 of the bougie 80 is made of latex-free, high-temperature silicone to aid in manipulation and placement and in forming and sizing the anastomosis. The bougie 80 also is radiopaque for x-ray verification. Because the bougie 80 is resistant to high temperatures and will not be harmed by a single episode of electro-cauterization, it is expected that the bougie 80 can be sterilized and reused a number of times, thereby reducing the cost of a given surgical procedure.

### Method of Operation

In operation, except as noted herein, the usual open or laparoscopic procedures are used to perform the gastric bypass surgical procedure. Either before or after the patient has been anesthetized, as appropriate, the tube 42 is intubated into the esophagus such that the tip 46 is disposed within the stomach 10, the small upper pouch 12, or in the region of the stoma 22, as the surgeon desires. By suitable use of the large syringe 54, the apparatus 40 can be used for purposes of evacuation, decompression, or lavage.

The method according to the invention includes the steps of positioning the tube 42 within the esophagus with the distal end of the tube 42 (the tip 46) downstream of the stoma 22. The method includes the step of occluding the Roux loop 24 downstream of the newly formed stoma 22 and the tip 46 by applying the clamp 90 at a desired location. The method includes the further step of establishing an airtight seal in the esophagus by inflating the balloon 58 and thereafter pumping air through the tube 42 by use of the syringe 54. The pressure is increased to a high level so that a good test of the integrity of the various sutures and staples is obtained. By turning the valve 64 to a shut position, air or other inflating fluid can be retained within the inflated balloon 58 so that the seal with the esophagus is maintained. Due to the simplicity and compactness of the apparatus 40, the surgeon can manipulate the syringe 66 and the valve 64 with one hand, if necessary or desired.

Although the invention has been described in its preferred form with a certain degree of particularity, it will be understood that the present disclosure of the preferred embodiments has been made only by way of example and that various changes may be resorted to without departing from the true spirit and scope of the invention as hereinafter claimed.

What is claimed is:

1. Surgical apparatus suitable for performing gastric bypass surgery, comprising:
a first tube made of a flexible material that defines a first passageway, the first tube having a tip end, a central portion, and an entrance/exit end, the tip end being provided with openings that establish fluid communication with the first passageway;
a first syringe that can be attached to the entrance/exit end;
an expansible balloon disposed adjacent the openings in the tip end and surrounding the first tube, the balloon being positioned intermediate the openings in the tip end and the central portion;
a second passageway extending along at least a portion of the length of the tube, the second passageway establishing fluid communication with the balloon;
a branch tube projecting from the side of the first tube in the region of the entrance/exit end, the branch tube being in fluid communication with the second passageway;
a shutoff valve disposed in the branch tube; and
a second syringe that can be attached to the shutoff valve.

2. The surgical apparatus of claim 1, wherein the tube includes a wall, and the second passageway is formed as part of the wall.

3. The surgical apparatus of claim 1, further comprising a second tube that is disposed within the first passageway and which defines the second passageway.

4. The surgical apparatus of claim 1, wherein the first tube is made of a material selected from the group consisting of latex, vinyl, and silicone.

5. The surgical apparatus of claim 1, further comprising a plurality of radiographic markers disposed at intervals along the length of the central portion.

6. The surgical apparatus of claim 5, wherein the radiographic markers are positioned approximately 10 cm from each other.

7. The surgical apparatus of claim 1, wherein the entrance/exit end is tapered, and further comprising an adapter having first and second ends, the first end being tapered for insertion into the entrance/exit end and the second end defining a luer slip connector to receive the first syringe.

8. The surgical apparatus of claim 1, wherein the connection between the shutoff valve and the second syringe is made by a luer lock connector.

9. Apparatus especially adapted for use in gastric bypass surgery, comprising a bougie having an elongate body portion and an enlarged tip, the tip being made of a material resistant to temperatures encountered during electro-cauterization.

10. The apparatus of claim 9, where the body portion and the tip are made of high-temperature silicone.

11. The apparatus of claim 9, wherein the tip is coated with a temperature-resisting substance such as TEFLON fluoropolymer.

12. A method of testing the integrity of an end-to-end anastomosis connection in a patient in which first and second conduits are joined, comprising the steps of:
occluding the first conduit upstream of the anastomosis connection;
occluding the second conduit downstream of the anastomosis connection;
pumping fluid into the occluded section;
increasing the pressure of the fluid to a desired level; and
checking the anastomosis connection for leaks.

13. The method of claim 12, in which the steps of occluding the first conduit upstream of the anastomosis connection, occluding the second conduit downstream of the anastomosis connection, pumping fluid into the occluded section, and increasing the pressure of the fluid to a desired level are performed by:
providing an esophageal-gastric tube having:
a first tube made of a flexible material that defines a first passageway, the first tube having a tip end, a central portion, and an entrance/exit end, the tip end being provided with openings that establish fluid communication with the first passageway;
a first syringe that can be attached to the entrance/exit end;
an expansible balloon disposed adjacent the openings in the tip end and surrounding the first tube, the balloon being positioned intermediate the openings in the tip end and the central portion;
a second passageway extending along at least a portion of the length of the tube, the second passageway establishing fluid communication with the balloon;
a branch tube projecting from the side of the first tube in the region of the entrance/exit end, the branch tube being in fluid communication with the second passageway;
a shutoff valve disposed in the branch tube; and
a second syringe that can be attached to the shutoff valve;
providing a clamp and applying the clamp to the second conduit;
inserting the esophageal-gastric tube into the esophagus of the patient;
inflating the balloon by pumping fluid into the second passageway by means of the second syringe until a fluid-tight seal between the balloon and the first conduit is established;
closing the shutoff valve; and
pumping fluid into the first passageway and out though the openings in the tip end by means of the first syringe until a desired pressure level in the occluded section is attained.

14. The method of claim 12, wherein the fluid is air.
